(19) United States
(12) Patent Application Publication
(10) Pub. No.: US 2020/0201338 A1
(43) Pub. Date: Jun. 25, 2020

(54) Hospital Automated Guided Vehicle and Method of Operating the Same

(71) Applicant: Harmonicareu co., Ltd., Hsinchu County (TW)

(72) Inventors: Min-Yang Wang, Hsinchu City (TW); Chin-Yi Hsieh, Taipei City (TW); Chen-Yang Kuo, Hsinchu County (TW)

(21) Appl. No.: 16/292,349

(22) Filed: Mar. 5, 2019

Related U.S. Application Data
(60) Provisional application No. 62/784,543, filed on Dec. 24, 2018.

(51) Int. Cl. G05D 1/02; G05D 1/00; G16H 10/60; G07C 9/00; H04W 4/80; H04W 4/40

(52) U.S. Cl. CPC G05D 1/0214; G05D 1/0088; H04W 4/40; G07C 9/00174; H04W 4/80; G16H 10/60

(57) ABSTRACT

A hospital automatic guided vehicle includes a vehicle housing, an object tracking sensor, a plurality of wheels and a controller. The object tracking sensor is mounted to the vehicle housing and used to track a location of a short-range wireless transmitter. The plurality of wheels are rotatably mounted to the vehicle housing and used to move the hospital automatic guided vehicle. The controller is coupled to the object tracking sensor, and used to control the plurality of wheels to move the hospital automatic guided vehicle according to the location of the short-range wireless transmitter.

### CROSS REFERENCE TO RELATED APPLICATIONS

This non-provisional application claims priority of U.S. provisional application No. 62/784,543, filed on Dec. 24, 2018, included herein by reference in its entirety.

### BACKGROUND OF THE INVENTION

### 1. Field of the Invention

The invention relates to robot technology, and specifically, to a hospital automated guided vehicle and a method of operating the same.

### 2. Description of the Prior Art

Medical carts are moved on wheels and positioned where needed in hospitals or other healthcare facilities. Often, the medical carts are equipped with various storage cabinets and hospital supplies to meet the diverse requirements for medical use, and therefore, the medical carts are heavy in weight and difficult to maneuver to and from bedsides in hospitals.

Automated guided vehicles (AGV) have been adopted in harsh environments such as industries or warehouses to load and transfer materials or goods by following markers or wires in the floor or using vision, magnets or lasers for navigation. However, AGVs have not been employed as medical carts in hospital environments where controlled drugs may be transported and security requirements for accessing the controlled drugs are high.

Thus, there arises a need for an AGV capable of automatically transferring medications and collecting healthcare information in the hospital while providing a well-controlled access to the contents thereof.

### SUMMARY OF THE INVENTION

In one aspect of the invention, a hospital automatic guided vehicle includes a vehicle housing, an object tracking sensor, a plurality of wheels and a controller. The object tracking sensor is mounted to the vehicle housing and used to track a location of a short-range wireless transmitter. The plurality of wheels are rotatably mounted to the vehicle housing and used to move the hospital automatic guided vehicle. The controller is coupled to the object tracking sensor, and used to control the plurality of wheels to move the hospital automatic guided vehicle according to the location of the short-range wireless transmitter.

In another aspect of the invention, a method of operating a hospital automatic guided vehicle is provided. The hospital automatic guided vehicle includes a vehicle housing, an object tracking sensor, a plurality of wheels and a controller. The method includes the object tracking sensor tracking a location of a short-range wireless transmitter, and the controller controlling the plurality of wheels to move the hospital automatic guided vehicle according to the location of the short-range wireless transmitter.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a system diagram of a hospital Automated Guided Vehicle (AGV) management system according to an embodiment of the invention.

FIG. 2 is a perspective view of a hospital automated guided vehicle according to an embodiment of the invention.

FIG. 3 is a block diagram of a hospital automated guided vehicle according to an embodiment of the invention.

FIG. 4 depicts operations of an intelligent tracking mode of the hospital automated guided vehicle in FIG. 1.

FIG. 5 depicts operations of a track guiding mode of the hospital automated guided vehicle in FIG. 1.

FIG. 6 depicts operations of a reservation mode of the hospital automated guided vehicle in FIG. 1.

FIG. 7 is a flowchart of an intelligent tracking method adopted by the hospital AGV in FIGS. 4, 5, 6.

FIG. 8 is a flowchart of a manual method adopted by the hospital AGV in FIGS. 4, 5, 6.

FIG. 9 is a flowchart of a track guiding method adopted by the hospital AGV in FIGS. 4, 5, 6.

FIG. 10 is a flowchart of a reservation method adopted by the hospital AGV in FIGS. 4, 5, 6.

### DETAILED DESCRIPTION

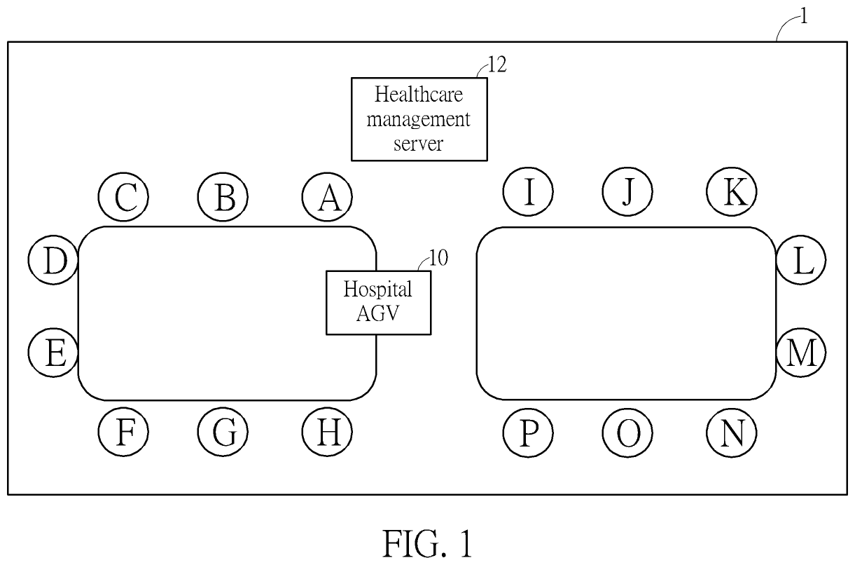

FIG. 1 is an exemplary floor plan of a hospital employing a hospital Automated Guided Vehicle (AGV) management system 1 according to an embodiment of the invention. The schematic floor plan comprises patient rooms A through P. The hospital AGV management system 1 comprises a hospital AGV 10 and a healthcare management server 12. The healthcare management server 12 may store healthcare data of patients in the hospital or manage assignments of the hospital AGV 10 and a responsible hospital attendant. When the hospital AGV 10 is assigned to a hospital attendant, only the assigned hospital attendant may operate the hospital AGV 10 or access the contents therein. The hospital AGV 10 may be moved from room to room, provide medications to patients, collect healthcare data of the patients and exchange healthcare data with the healthcare management server 12 via wireless connections.

Although only one hospital AGV 10 is shown in FIG. 1, it should be understood that more than one hospital AGVs 10 may be incorporated in the hospital AGV management system 1, and the healthcare data may be shared between the hospital AGVs in the hospital AGV management system 1.

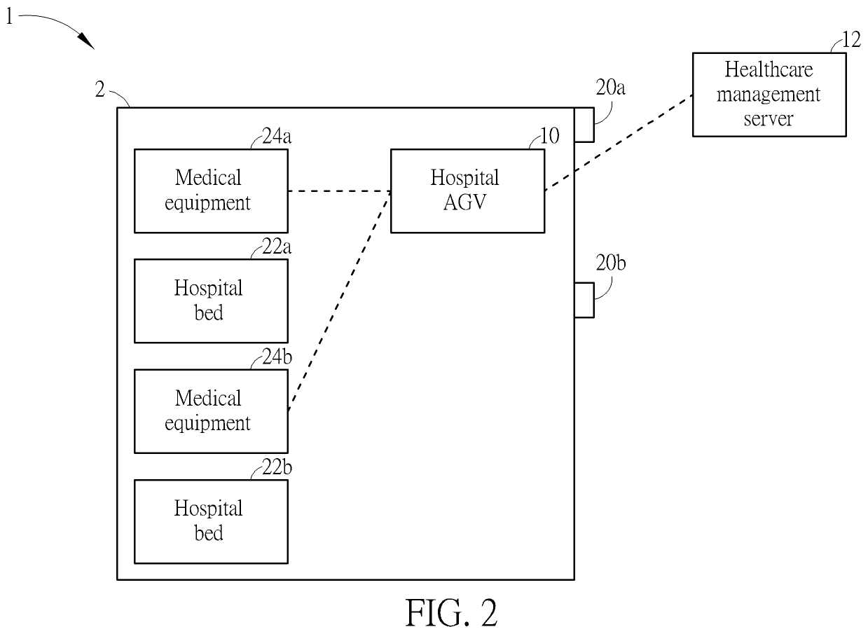

FIG. 2 is an exemplary room layout of a patient room 2 employing the hospital AGV management system 1 in FIG. 1. The patient room 2 includes positioning devices 20a, 20b, hospital beds 22a, 22b and medical equipment 24a, 24b. The medical equipment 24a, 24b may be positioned next to the hospital beds 22a, 22b to take medical measurements of patients on the hospital beds 22a, 22b, and may be medical monitors such as electrocariography (ECG) monitors, electroencephalography (EEG) monitors, blood pressure monitors or blood sugar monitors. The positioning devices 20a, 20b may be installed at two sides of the doorway of the patient room 2, and may provide location information uniquely identifying the patient room 2.

When the hospital AGV 10 is moved close to the patient room 2, the hospital AGV 10 may detect location information from at least one of the positioning devices 20a, 20b, identify the location of the hospital AGV 10 as being close to the patient room 2 using the location information, determine patients in the patient room 2, establish wireless connections with the medical equipment 24a, 24b and/or the healthcare management server 12, receive healthcare data of the patients in the patient room 2 from the medical equipment 24a, 24b, from the healthcare management server 12, or from a user input, and transmit the healthcare data from the medical equipment 24a, 24b to the healthcare management server 12 or other hospital AGVs for data backup. The healthcare data from the medical equipment 24a, 24b may be blood pressures, blood oxygen levels, heart rates, body temperatures, blood sugars, or uterine concentrations of the patients. The healthcare data from the healthcare management server 12 may be medical orders, medical records or healthcare data previously backed up. The healthcare data from the user input may be a medical order or a Kardex record entered by the hospital attendant, or a record of operations performed by the assigned hospital attendant. The positioning devices 20a, 20b may be passive tagged device such as radio frequency identifications (RFID), barcodes or other identification markers representing the patient room 2 and identifiable by the hospital AGV 10. In such a case, since the range of obtaining a clear identification of the passive tagged device is very limited, e.g., for scanning an RFID using the near field communication technology, the distance between the hospital AGV 10 and the RFID may be less than 20 cm, the location of the hospital AGV 10 may be approximated to the location the patient room 2. The positioning devices 20a, 20b may also be active signal transmission devices such as beacon transmitters regularly broadcasting beacon signals to be picked up by the hospital AGV 10 to identify the location of the hospital AGV 10 accordingly. In such a case, the location of the hospital AGV 10 may be estimated according to signal intensity of the beacon signals, and beacon signals having the strongest signal intensity indicate the closest proximity to the patient room 2.

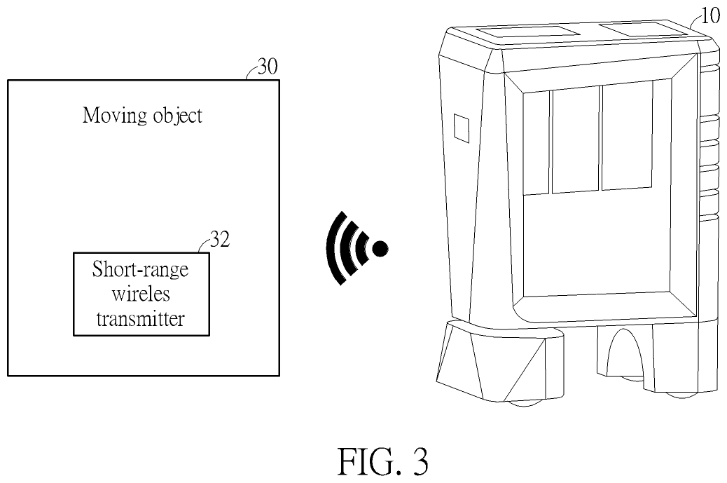

The hospital AGV 10 may be moved manually or automatically. In particular, the hospital AGV 10 may provide four operating modes, namely a manual mode, an intelligent tracking mode, a track guiding mode and a reservation mode. In the manual mode, the hospital AGV 10 may move according to an instruction given by a hospital attendant assigned to the hospital AGV 10. The instruction may be a voice instruction, a gesture, or a data instruction. In the intelligent tracking mode, the hospital AGV 10 may move automatically by tracking a moving object as shown in FIG. 3, where a short-range wireless transmitter 32 may be attached to a moving object 30 such as a hospital attendant and may transmit a short-range wireless signal at regular intervals as the moving object 30 advances, and the hospital AGV 10 may detect the short-range wireless signal to determine the location of the moving object 30 and the short-range wireless transmitter 32, thereby navigating the hospital AGV 10 to track behind the moving object 30 according to the location of the moving object 30 and the short-range wireless transmitter 32. The short-range wireless transmitter 32 may be an infrared transmitter, a visible light communication (VLC) transmitter, an optical transmitter, a Bluetooth transmitter, a Wi-Fi transmitter, or an ultrasonic transmitter, and may be implemented as a wearable device or a mobile device. In the track guiding mode, the hospital AGV 10 may move by following a programmed path. In the reservation mode, the hospital AGV 10 may move by traveling straight towards a target location. The programmed path and the target location may be set from the hospital AGV 10 or the healthcare management server 12. For example, when the track guiding mode is selected, the programmed path may be set based on the floor plan in FIG. 1 to follow the order of Rooms A through H and then Rooms I through P, and the hospital AGV 10 may be navigated in the hospital by following the programmed path; when the hospital AGV 10 advances from Room A to Room B according to the programmed path, the reservation mode may be selected and a target location of Room G may be entered, the hospital AGV 10 may be navigated towards Room G directly without passing Rooms B through F.

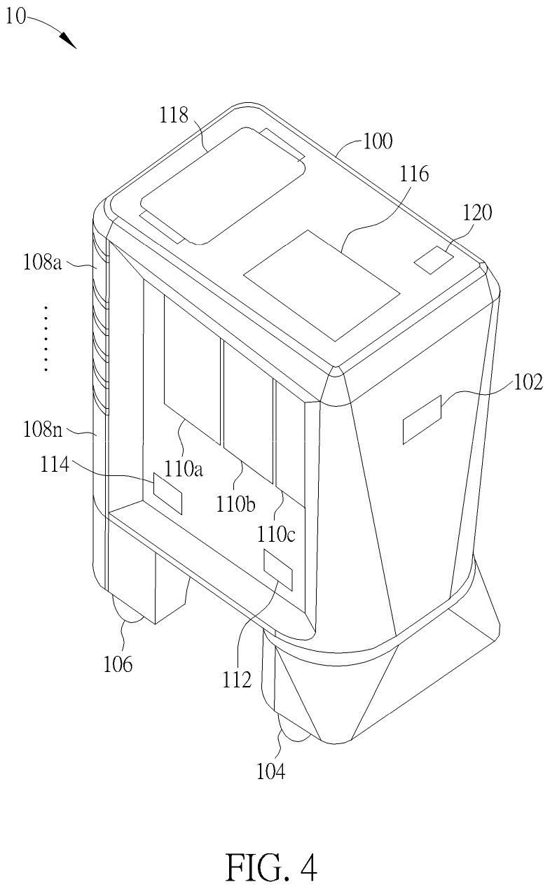

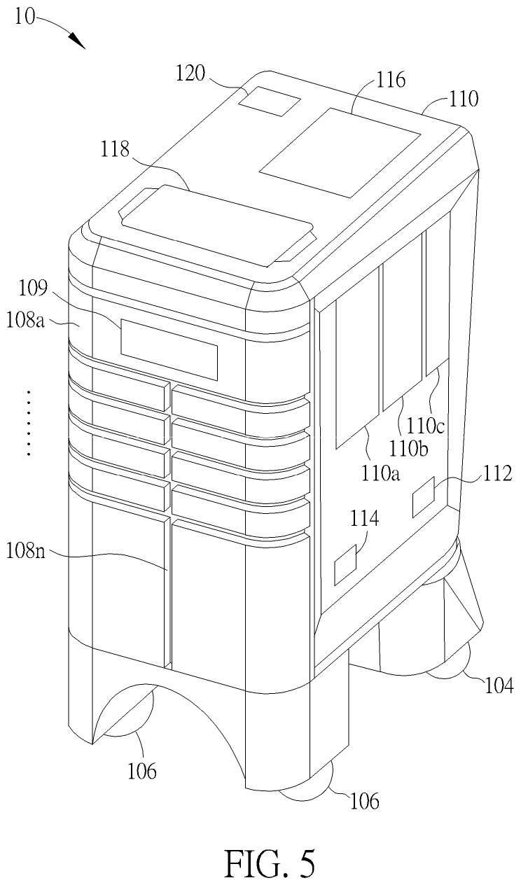

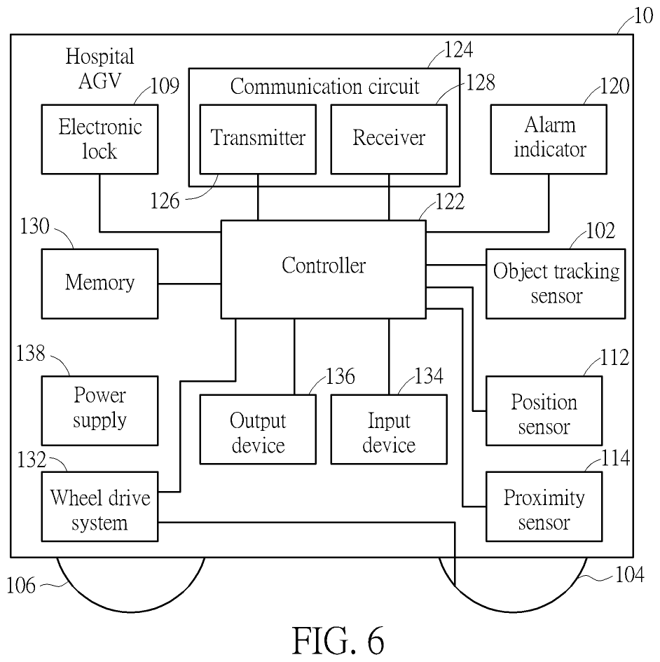

FIG. 4 and FIG. 5 are two perspective views of the hospital AGV 10, and FIG. 6 is a block diagram of the hospital AGV 10. The hospital AGV 10 comprises a vehicle housing 100, an object tracking sensor 102, a plurality of wheels 104, 106, a plurality of drawers 108a through 108n, an electronic lock 109, a plurality of medical waste disposal bins 110a through 110c, a position sensor 112, a proximity sensor 114, a tablet computer 116, an operating tray 118, an alarm indicator 120, a controller 122, a communication circuit 124 comprising a transmitter 126 and a receiver 128, memory 130, a wheel drive system 132, an input device 134, a display device 136 and a power supply 138.

The object tracking sensor 102 may be mounted to a front wall of the vehicle housing 100. The position sensor 112 and the proximity sensor 114 may be mounted to a sidewall of the vehicle housing 100 to respectively detect the location and the space for movement of the hospital AGV 10. The alarm indicator 120 may be mounted to a top wall of the vehicle housing 100 to indicate that an obstacle is blocking the way of the hospital AGV 10. The operating tray 118 may be detacheably mounted to the top wall of the vehicle housing 100, and the tablet computer 116 may be detacheably and rotatably mounted to the top wall of the vehicle housing 100. The plurality of wheels 104, 106 comprise front wheels 104 and rear wheels 106 rotatably mounted to a bottom wall of the vehicle housing 100 to assist in moving the hospital AGV 10 from location to location. The plurality of drawers 108a through 108n may be slidably arranged at a rear sidewall of the vehicle housing 100. The electronic lock 109 may be attached to the plurality of drawers 108a through 108n to respectively control locking and unlocking of the plurality of drawers 108a through 108n. The plurality of medical waste disposal bins 110a through 110c may be slidably arranged at the sidewall of the vehicle housing 100.

The controller 122 maybe coupled to the object tracking sensor 102, the electronic lock 109, the position sensor 112, the proximity sensor 114, the alarm indicator 120, the transmitter 126, the receiver 128, the memory 130, the wheel drive system 132, the input device 134 and the display device 136 to control operations thereof. The power supply 138 may be arranged in the hospital AGV 10 and may be coupled to all of the electronic components in the hospital AGV 10 to supply power thereto. The wheel drive system 132 is coupled to the front wheels 104. The controller 122, the transmitter 126, the receiver 128, the memory 130, the wheel drive system 132, and the power supply 138 may be located inside the vehicle housing 100.

In the intelligent tracking mode, the object tracking sensor 102 may be enabled to track the location of the moving object 30 carrying the short-range wireless transmitter 32 as depicted in FIG. 3, and the controller 122 may control the plurality of wheels 104, 106 via the wheel drive system 132 to move the hospital AGV 10 according to the location of the short-range wireless transmitter 32. The object tracking sensor 102 may adopt the signal transmission technology matching the short-range wireless transmitter 32, and may be an infrared sensor, a VLC (visible light communication) signal sensor, other optical signal sensor, a Bluetooth sensor, a Wi-Fi sensor, or an ultrasonic sensor.

The plurality of drawers 108a through 108n may be respectively adjustable in width, height and depth to customize the drawer spaces and fit the storage needs, and may be respectively arranged in closed position or open position. Since the plurality of drawers 108a through 108n may contain restricted medicines or controlled drugs, accesses to the plurality of drawers 108a through 108n are strictly controlled. When in the closed position, the plurality of drawers 108a through 108n may be locked by the electronic lock 109. The controller 122 may set the permission for a tagged device containing a unique identifier to access each of the drawers 108a through 108n. The tagged device may be an RFID, a barcode or other identification markers, and may be integrated into a wearable device or a mobile device. For arranging a drawer to the open position, the electronic lock 109 may scan the identifier from the tagged device, and when the identifier is identified as a tagged device authorized to access a corresponding drawer in the drawers 108a through 108n, the electronic lock 109 may unlock the corresponding drawer and then the corresponding drawer may be slid to the open position. In some embodiments, the tagged device maybe carried by a hospital attendant assigned to the hospital AGV 10 and contain an identification number designated to the assigned hospital attendant. In other embodiments, the tagged device may be carried by a patient having a doctor's prescription including medication matching the content of the corresponding drawer, and may contain an identification number of the specific patient. Each drug may be tagged with an RFID, barcode or machine identifiable marker. The memory 130 may keep a list of medication inventory in the hospital AGV 10. When the drug is removed from the drawer, the hospital AGV 10 may automatically remove the tagged number of the drug from the list of medication inventory, and the transmitter 126 may regularly transmit the updated list of medication inventory to the healthcare management server 12 and/or other hospital AGVs 10.

In addition to the permission of accesses to the plurality of drawers 108a through 108n, the controller 122 may also set or reset the permission of operating the hospital AGV 10. Prior to using the hospital AGV 10, the hospital AGV 10 may be configured to scan by the tablet computer 116 a tagged device designated to a user, and determine by the controller 122 whether the user has the permission to operate the hospital AGV 10 such as moving the AGV 10, entering data into records on the hospital AGV 10, entering instructions to the hospital AGV 10, accessing remote data on a remote server or other hospital AGP 10, and performing other operations on the AGV 10. Accordingly, the controller 122 may set permission of operating the hospital AGV 10 for a first tagged device carried by a first hospital attendant assigned to the hospital AGV 10. The first tagged device may contain a first identifier uniquely identifying the first hospital attendant. Further, the assignment of the hospital AGV 10 may be reset and transferred to another hospital attendant. In the reset process, the receiver 128 may receive a permission reset instruction indicating a request for resetting the permission of operating the hospital AGV 10 to a second tagged device. The second tagged device may be carried by a second hospital attendant and contain a second identifier uniquely identifying the second hospital attendant. Next, the input device 134 may receive a confirmation instruction indicating a confirmation of resetting the permission of operating the hospital AGV 10 to the second tagged device, and the controller 122 may cancel the assignment of the first hospital attendant and assign the second hospital attendant to the hospital AGV 10, allowing the second hospital attendant to control the operations of the hospital AGV 10 and preventing the first hospital attendant from continuing using the hospital AGV 10. The first tagged device and the second tagged device may be wearable devices or mobile devices, and the first identifier and the second identifier may each be an RFID, a barcode or other identification markers.

The proximity sensor 114 may be an ultrasonic sensor, and may be employed to prevent the hospital AGV 10 from colliding with any obstacle blocking the path when the hospital AGV 10 maneuvers in the hospital. In particular, the proximity sensor 114 may detect a space for movement of the hospital AGV 10. In some embodiments, if the space for movement of the hospital AGV 10 is less than a predetermined space such as 5 cm, the alarm indicator 120 may be turned on to notify the hospital attendant of the limited space for movement, the controller 122 may control at least one of the plurality of wheels 104, 106 to rotate by an angle such as 10° and proceed to move the hospital AGV 10. In other embodiments, if the space for movement of the hospital AGV 10 is less than the predetermined space, the alarm indicator 120 maybe turned on to notify the hospital attendant of the limited space for movement, the controller 122 may control the plurality of wheels 104, 106 to remain stationary until the obstacle is removed from the path and the space for movement of the hospital AGV 10 exceeds the predetermined space. The alarm indicator 120 maybe an indicator light, a buzzer or a speaker.

The input device 134 may be a video camera, a microphone, a touchscreen, a tablet computer, a keyboard, a mouse, a keypad, a joystick, a wireless remote control, a wired remote control, or other input devices. The display device 136 may be a monitor or a tablet computer. The input device 134 and the display device 136 may be integrated into the tablet computer 116. The operating tray 118 is a working space for a hospital attendant to prepare medications for patients and perform medical procedures, and may be detached from the hospital AGV 10 and transferred to the bedside of the patient upon completion of medication preparation or other medical procedures.

The plurality of medical waste disposal bins 110a through 110c may respectively contain various types of medical wastes such as sharp wastes, biohazardous wastes, chemical wastes, pharmaceutical wastes, radioactive wastes, and unregulated wastes. For example, the sharp wastes may be needles, ampules, broken glass, blades, razors, or staples; the biohazardous wastes may be infective wastes, blood contaminated wastes, contaminated personal protective equipment, or biological cultures and stocks; the chemical wastes maybe used vials or ampules, used syringes or needles, used intravenous tubing, used personal protective equipment or used packaging; the pharmaceutical wastes may be pills, injectables or antibiotics; the radioactive wastes may be radioactive substances; and the unregulated wastes are all the other wastes produced at the hospital. Each of the plurality of medical waste disposal bins 110a through 110c may be slid out or tilted out to an open position and slid in or returned to a closed position for achieving easy disposal of medical wastes. Further, each of the plurality of medical waste disposal bins 110a through 110c may be replaceable by an empty replacement disposal bin to prevent a hospital attendant from being in direct contact with the contaminated contents.

The hospital AGV 10 may perform a data acquisition process upon arriving a patient room. Specifically, the position sensor 112 may determine the location of the hospital AGV 10 by receiving the location information from the positioning devices positioned at the doorway of each patient room, and the receiver 128 may receive the healthcare data according to the location of the hospital AGV 10 and then store the healthcare data in the memory 130. When the hospital AGV 10 approaches the doorway of a patient room, the controller 122 may determine that its location is in the proximity to the patient room and acquire a list of patients in the patient room either locally or from the healthcare management server 12, the receiver 128 may download the healthcare data associated with the patients from medical equipment and store the downloaded healthcare data locally in the memory 130, or the transmitter 126 may transmit the downloaded healthcare data to the healthcare management server 12 or other hospital AGVs. In some embodiment, the receiver 128 may also download the healthcare data associated with the patients, such as medical orders or medical records of the patients, from the healthcare management server 12, and the display device 136 may display thereon the downloaded healthcare data for the healthcare attendant to quickly know medical conditions of the patients.

The hospital AGV 10 may be used to access inventory data of another hospital AGV 10 such as the list of medication inventory as discussed in the preceding paragraph. During an inventory inquiry process, the receiver 128 may receive an inventory data of another hospital AGV 10, and the display device 136 may display the inventory data of the other hospital AGV 10.

The wheel drive system 132 may comprise an engine and a motor to drive the front wheels 104. The rear wheels 106 do not receive power from the wheel drive system 132 but rotate with the rotations of the front wheels 104. Nevertheless, the invention is not limited to the front-wheel drive configuration, rear-wheel drive and all-wheel drive configurations may also be implemented in the hospital AGV 10.

The power supply 138 may be a field-replaceable or rechargeable battery, and may be charged by wireless power transfer technologies such as inductive coupling, capacitive coupling, magnetodynamic coupling, microwaves and optical coupling.

Although only one proximity sensor 114 is shown on FIGS. 4 and 5, it should be understood that any plural number of proximity sensors may be incorporated in the hospital AGV 10 to monitor the spaces around the hospital AGV 10 for movement of the hospital AGV 10.

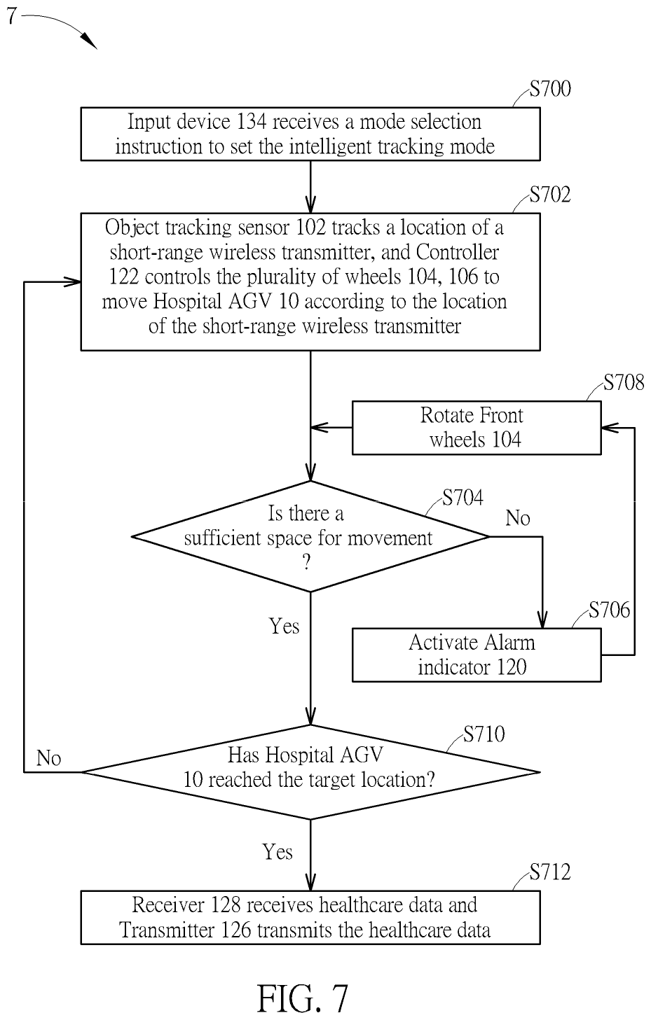

FIG. 7 is a flowchart of an intelligent tracking method 7 adopted by the hospital AGV 10. The intelligent tracking method 7 comprises Steps S700 through S712, wherein Steps S700 and S702 are used to configure the hospital AGV 10 to move automatically by tracking a moving object. Steps S704 through S708 are used to provide collision avoidance, and Step S710 and S712 are used to perform data acquisition. Any reasonable technological change or step adjustment is within the scope of the disclosure. Steps S700 through S712 are detailed as below:

Step S700: Input device 134 receives a mode selection instruction to set the intelligent tracking mode;

Step S702: Object tracking sensor 102 tracks a location of a short-range wireless transmitter, and Controller 122 controls the plurality of wheels 104, 106 to move Hospital AGV 10 according to the location of the short-range wireless transmitter;

Step S704: Is there a sufficient space for movement? If so, go to Step S706, and if not, go to Step S710;

Step S706: Activate Alarm indicator 120;

Step S708: Rotate the front wheels 104; go to Step S704

Step S710: Has Hospital AGV 10 reached the target location?If so, go to Step S712, and if not, go to Step S702;

Step S712: Receiver 128 receives healthcare data and Transmitter 126 transmits the healthcare data.

The intelligent tracking method 7 is used to switch the operating mode of the hospital AGV 10 to the intelligent tracking mode, a hospital attendant authorized to operate the hospital AGV 10 may select the intelligent tracking mode by entering a mode selection instruction into the input device 134. Correspondingly in Step S700, the input device 134 receives the mode selection instruction indicating that the intelligent tracking mode is selected, and the controller 122 sets the operating mode to the intelligent tracking mode and activates the object tracking sensor 102 for object tracking. The hospital attendant may carry a short-range wireless transmitter and lead the way in front of the object tracking sensor 102. In Step S702, the object tracking sensor 102 is enabled to track the location of the short-range wireless transmitter and hence track the location of the hospital attendant, and the controller 122 steers the plurality of wheels 104, 106 via the wheel drive system 132 to move the hospital AGV 10 by following the location of the hospital attendant. During tracking the hospital attendant, the proximity sensor 114 regularly detects the space for movement, and in Step S704, the controller 122 determines whether a sufficient space for movement is available by comparing the detected space to a minimum space limit, when the space exceeds the minimum space limit, determine that the space for movement is sufficient; when the space is less than or equal to the minimum space limit, determine that the space for movement is insufficient. When there is no sufficient space for movement, the alarm indicator 120 notifies the hospital attendant of the insufficient space in Step S706 by flashing a warning light, playing a warning tone or showing a warning sign, the controller 120 steers the front wheels 104 to rotate by an angle via the wheel drive system 132 in Step S708, and the intelligent tracking method 7 goes back to Step S704 to determine once again whether there is sufficient space for movement. When there is a sufficient space for movement, in Step S710, the controller 122 then determines whether the hospital AGV 10 has reached a target location according to the location of the hospital AGV 10 determined by the position sensor 112. The target location may be a patient room, a medication storage area or any area of interest in the hospital. When the hospital AGV 10 has reached the target location, in Step S712, the receiver 128 receives healthcare data of the patients from the healthcare management server 12 or the medical equipment, the input device 134 receives user input healthcare data as discussed in FIG. 2, and the transmitter 126 uploads the healthcare data to the healthcare management server 12 and/or other hospital AGVs 10, as discussed in the preceding paragraphs of the disclosure. When the hospital AGV 10 has not yet reached the target location, the hospital AGV 10 continues to follow the hospital attendant in Step S702, and Steps S702 through S710 are performed until the target location is reached.

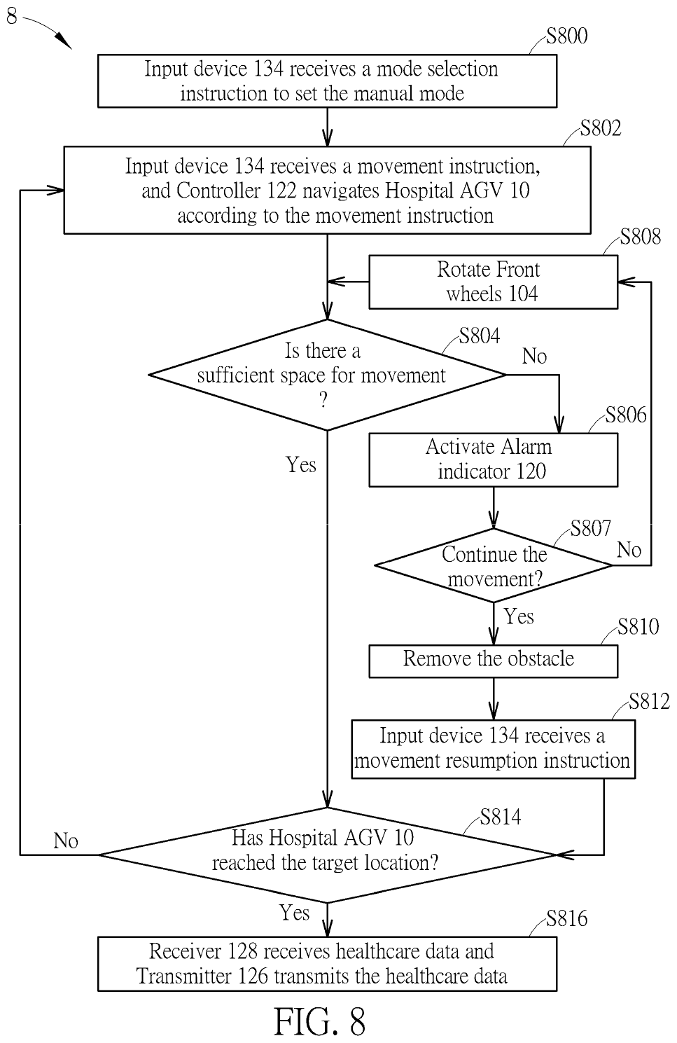

FIG. 8 is a flowchart of the manual method 8 adopted by the hospital AGV 10. The manual method 8 comprises Steps S800 through S816, wherein Steps S800 and S802 configure the hospital AGV 10 to move according to instruction given by a hospital attendant authorized to use the hospital AGV 10. Steps S804 through S812 are used to provide collision avoidance, and Step S814 and S816 are used to perform data acquisition. Any reasonable technological change or step adjustment is within the scope of the disclosure. Steps S800 through S816 are detailed as below:

Step S800: Input device 134 receives a mode selection instruction to set the manual mode;

Step S802: Input device 134 receives a movement instruction, and Controller 122 navigates Hospital AGV 10 according to the movement instruction;

Step S804: Is there a sufficient space for movement? If so, go to Step S814, and if not, go to Step S806;

Step S806: Activate Alarm indicator 120;

Step S807: Continue the movement? If so, go to Step S810, and if not, go to Step S808;

Step S808: Rotate the front wheels 104; go to Step S804;

Step S810: Remove the obstacle;

Step S812: Input device 134 receives a movement resumption instruction;

Step S814: Has Hospital AGV 10 reached the target location? If so, go to Step S816, and if not, go to Step S802;

Step S816: Receiver 128 receives healthcare data and Transmitter 126 transmits the healthcare data.

The manual method 8 is used to switch the operating mode of the hospital AGV 10 to the manual mode, a hospital attendant authorized to operate the hospital AGV 10 may select the manual mode by entering a mode selection instruction into the input device 134. Correspondingly in Step S800, the input device 134 receives the mode selection instruction indicating that the manual mode is selected, and the controller 122 sets the operating mode to the manual mode to move the hospital AGV 10 according to manually input instructions. In Step S802, the input device 134 receives a movement instruction such as moving straight forward, turning left or right or reversing, and the controller 122 navigates the hospital AGV 10 according to the movement instruction. In Step S804, the controller 122 determines whether a sufficient space for movement is available as discussed in Step S704 in FIG. 7. When there is no sufficient space for movement, in Step S806, the alarm indicator 120 notifies the hospital attendant of the insufficient space as discussed in Step S706 in FIG. 7. In Step S807, the controller 122 prompts the hospital attendant to enter whether to continue the movement of the hospital AGV 10 according to the direction indicated in the movement instruction. When the hospital attendant rejects to continue the movement, the controller 120 steers the front wheels 104 to rotate by an angle via the wheel drive system. 132 in Step S808, and intelligent tracking method 7 goes back to Step S804 to determine once again whether there is sufficient space for movement. In Step S810, when the hospital attendant confirms to continue the movement, the hospital AGV 10 may actively remove the obstacle on the path of movement, or passively remains still until the obstacle on the path is cleared. After the proximity sensor 114 detects that the obstacle has been cleared, the controller 122 prompts the hospital attendant to further enter a movement resumption instruction. In Step S812, the input device 134 receives a movement resumption instruction indicating to resume the movement of the hospital AGV 10. Next in Step S814, the controller 122 determines whether the hospital AGV 10 has reached a target location according to the location of the hospital AGV 10 determined by the position sensor 112. When the hospital AGV 10 has reached the target location, in Step S816, the receiver 128 acquires healthcare data of the patients from the healthcare management server 12 or the medical equipment, the input device 134 receives user input healthcare data as discussed in FIG. 2, and the transmitter 126 uploads the healthcare data to the healthcare management server 12 and/or other hospital AGVs 10, as discussed in the preceding paragraphs. When the hospital AGV 10 has not yet reached the target location, in Step S802, the hospital AGV 10 continues to move according to another movement instruction given by the hospital attendant, and Steps S802 through S814 are performed until the target location is reached.

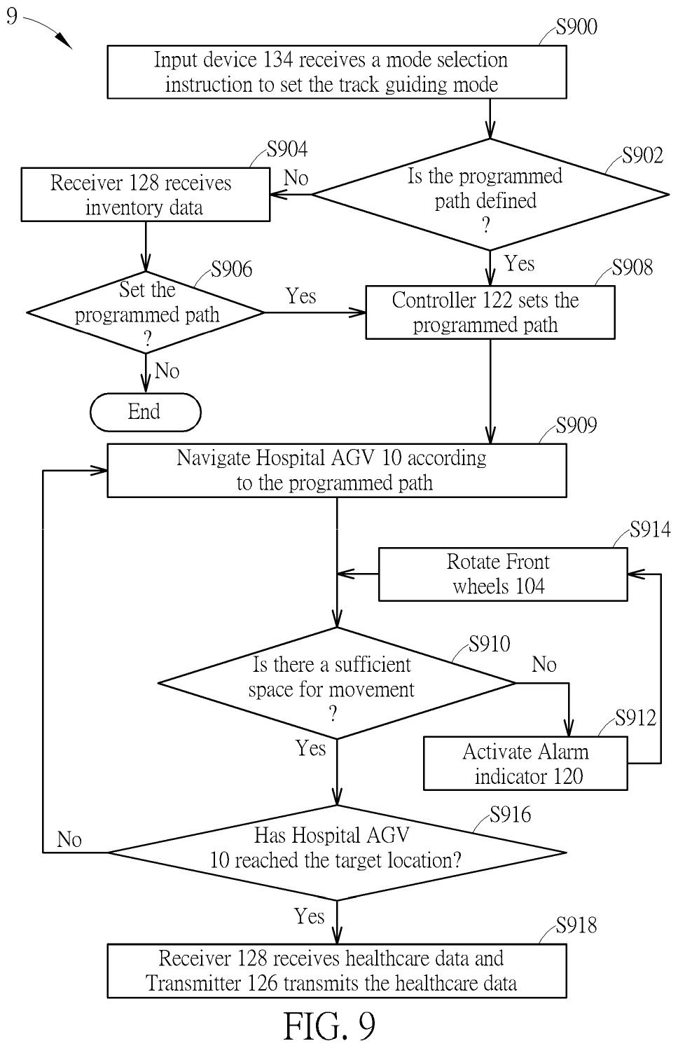

FIG. 9 is a flowchart of a track guiding method 9 adopted by the hospital AGV 10. The track guiding method 9 comprises Steps S900 through S918, wherein Steps S900 through S909 are used to configure the hospital AGV 10 to move automatically according to a programmed path. Steps S910 through S914 are used to provide collision avoidance, and Step S916 and S918 are used to perform data acquisition. Any reasonable technological change or step adjustment is within the scope of the disclosure. Steps S900 through S918 are detailed as below:

Step S900: Input device 134 receives a mode selection instruction to set the track guiding mode;

Step S902: Is the programmed path defined? If so, go to Step S908, and if not, go to Step S904;

Step S904: Receiver 128 receives inventory data;

Step S906: Set the programmed path? If so, go to Step S908, and if not, end;

Step S908: Controller 122 sets the programmed path;

Step S909: Navigate Hospital AGV 10 according to the programmed path;

Step S910: Is there a sufficient space for movement? If so, go to Step S916, and if not, go to Step S912;

Step S912: Activate Alarm indicator 120;

Step S914: Rotate the front wheels 104; go to Step S910;

Step S916: Has Hospital AGV 10 reached the target location? If so, go to Step S918, and if not, go to Step S909;

Step S918: Receiver 128 receives healthcare data and Transmitter 126 transmits the healthcare data.

Prior to operating, an operating area may be defined for each hospital AGV 10 by manually configuring an area on a floor plan from the healthcare management server 12 or the hospital AGV 10, or setting up a boundary within the floor plan using boundary markers such as magnetic strips or IR beam generators. A target location or a path is referred to as defined when the target location or the path are located inside the defined operating area.

The hospital AGV 10 may only move within the defined operating area or the boundary, and may not move forwards when the hospital AGV 10 detects an insufficient space for movement. The hospital AGV 10 may compute a traveling path within the operating area or the boundary according to the programmed path or the target location, and move accordingly. For example, the hospital AGV 10 may compute the shortest path from the current location to a target location, and move according to the shortest path. In another example, when the front path is blocked, the hospital AGV 10 may compute an alternative path to rejoin the programmed path from the current location, and then move according to the alternative path.

The track guiding method 9 is used to switch the operating mode of the hospital AGV 10 to the track guiding mode. A hospital attendant authorized to operate the hospital AGV 10 may select the track guiding mode by entering a mode selection instruction into the input device 134. Correspondingly in Step S900, the input device 134 receives the mode selection instruction indicating that the track guiding mode is selected and a programmed path for the hospital AGV 10 to follow, and in Step S902, the controller 122 determine whether all locations on the programmed path are defined. For example, if the second floor of a hospital building has been defined for the hospital AGV 10, the programmed path is regarded as defined if all locations on the programmed path are on the second floor, and the programmed path is regarded as undefined if one or more locations on the programmed path are located on a different floor of the hospital building such as the first floor. When all locations on the programmed path are defined, the controller 122 sets the programmed path in Step S908 and the controller 122 navigates movements of the hospital AGV 10 according to the programmed path in Step S909. In Step S904, when one or more locations on the programmed path are undefined, the receiver 128 receives inventory data of another hospital AGV 10, and the display device 136 displays the inventory data of the other hospital AGV 10 and the current hospital AGV 10. And in Step S906, the controller 122 determines whether to set the programmed path either by a user input or by determining whether a criterion of setting the programmed path is met. For example, if the inventory data shows that the current hospital AGV 10 contains more required medication inventory than the other hospital AGV 10, the hospital attendant may enter an instruction into the input device 134 to accept the undefined programmed path, otherwise the hospital attendant may enter an instruction into the input device 134 to reject the undefined programmed path. Alternatively, the controller 122 may determine to use the undefined programmed path to navigate the current hospital AGV 10 if the inventory data shows that the current hospital AGV 10 contains more required medication inventory than that in other hospital AGVs 10, and otherwise the controller 122 may reject the undefined programmed path. If it is determined to set the programmed path, the controller 122 sets the programmed path in Step S908, and navigates the current hospital AGV 10 according to the programmed path in Step S909. If it is determined not to set the programmed path, the track guiding method 9 is exited.

Steps S910 through S918 are similar to Steps S704 through S712, and explanation therefor is omitted for brevity.

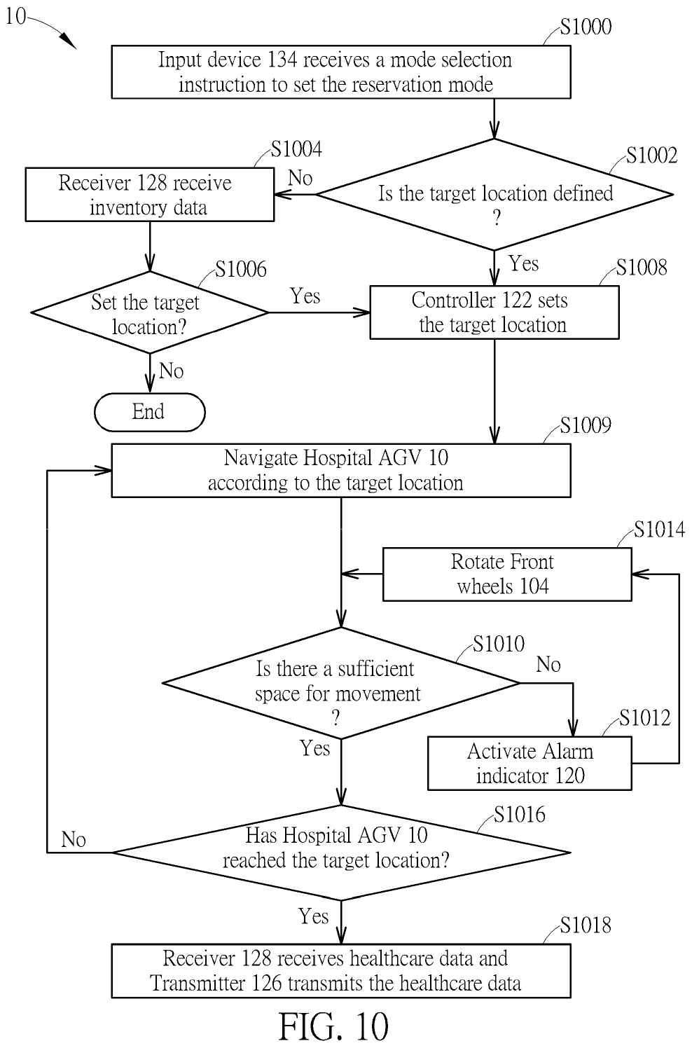

FIG. 10 is a flowchart of a reservation method 10 adopted by the hospital AGV 10. The reservation method 10 comprises Steps S1000 through S1018, wherein Steps S1000 through S1009 are used to configure the hospital AGV 10 to move automatically according to a target location, Steps S1010 through S1014 are used to provide collision avoidance, and step S1016 and S1018 are used to perform data acquisition. Any reasonable technological change or Step adjustment is within the scope of the disclosure. Steps S1000 through S1018 are detailed as below:

Step S1000: Input device 134 receives a mode selection instruction to set the reservation mode;

Step S1002: Is the target location defined? If so, go to Step S1008, and if not, go to Step S1004;

Step S1004: Receiver 128 receives inventory data;

Step S1006: Set the target location? If so, go to Step S1008, and if not, end;

Step S1008: Controller 122 sets the target location;

Step S1009: Navigate Hospital AGV 10 according to the target location;

Step S1010: Is there a sufficient space for movement? If so, go to Step S1016, and if not, go to Step S1012;

Step S1012: Activate Alarm indicator 120;

Step S1014: Rotate the front wheels 104; go to Step S1010;

Step S1016: Has Hospital AGV 10 reached the target location? If so, go to Step S1018, and if not, go to Step S1009;

Step S1018: Receiver 128 receives healthcare data and Transmitter 126 transmits the healthcare data.

The reservation method 10 is similar to track guiding method 9 except that rather than a programmed path a target location is set for the hospital AGV 10. The target location may be set by adding the target location to an existing programmed path on the hospital AGV 10. The priority of the newly added target location may be highest among all the target locations on the programmed path, or the priorities of all the target locations may be defined according to the current location of the hospital AGV 10, with the closest target location having the highest priority and the farthest target location having the lowest priority. Steps S1000 through S1018 are similar to Steps S900 through S918, and explanation therefor is omitted for brevity.

The hospital AGVs and methods of operating the same as provided in FIGS. 1 through 10 can be used to automatically transfer medications and collect healthcare information in a hospital environment while providing a well-controlled access to the contents of the hospital AGVs.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A hospital automatic guided vehicle comprising:
a vehicle housing;
an object tracking sensor, mounted to the vehicle housing and configured to track a location of a short-range wireless transmitter;
a plurality of wheels, rotatably mounted to the vehicle housing and configured to move the hospital automatic guided vehicle; and
a controller, coupled to the object tracking sensor, and configured to control the plurality of wheels to move the hospital automatic guided vehicle according to the location of the short-range wireless transmitter.

2. The hospital automatic guided vehicle of claim 1, further
comprising a drawer and an electronic lock attached thereon;
wherein the controller is further configured to set a permission to access the drawer for a tagged device containing an identifier; and
the electronic lock is configured to scan the identifier from the tagged device and unlock the drawer when the identifier is identified as a tagged device authorized to access the drawer.

3. The hospital automatic guided vehicle of claim 1, further comprising:
a position sensor, mounted to the vehicle housing and configured to determine a location of the hospital automatic guided vehicle;
memory; and
a receiver, coupled to the position sensor and the memory, and configured to receive healthcare data according to the location of the hospital automatic guided vehicle and store the healthcare data in the memory.

4. The hospital automatic guided vehicle of claim 3, wherein the healthcare data is downloaded from a remote server and is associated with a patient close to the location of the hospital automatic guided vehicle.

5. The hospital automatic guided vehicle of claim 3, wherein the healthcare data is downloaded from medical equipment and is associated with a patient close to the location of the hospital automatic guided vehicle.

6. The hospital automatic guided vehicle of claim 3, further comprising a transmitter coupled to the memory and configured to transmit the healthcare data to a remote server.

7. The hospital automatic guided vehicle of claim 1, further comprising a proximity sensor, mounted to a sidewall of the vehicle housing, coupled to the controller and configured to detect a space for movement of the hospital automatic guided vehicle.

8. The hospital automatic guided vehicle of claim 7, wherein when the space for movement of the hospital automatic guided vehicle is less than a predetermined space, the controller is configured to control at least one of the plurality of wheels to rotate by an angle and proceed to move the hospital automatic guided vehicle.

9. The hospital automatic guided vehicle of claim 7, wherein when the space for movement of the hospital automatic guided vehicle is less than a predetermined space, the controller is configured to control the plurality of wheels to remain stationary until the space for movement of the hospital automatic guided vehicle exceeds the predetermined space.

10. The hospital automatic guided vehicle of claim 1, wherein the controller is further configured to set a permission of operating the hospital automatic guided vehicle for a first tagged device containing a first identifier.

11. The hospital automatic guided vehicle of claim 10, further comprising:
a receiver, coupled to the controller, and configured to receive a permission reset instruction indicating a request for resetting the permission of operating the hospital automatic guided vehicle to a second tagged device containing a second identifier; and
an input device, detacheably connected to the vehicle housing, coupled to the controller, and configured to receive a confirmation instruction indicating a confirmation of resetting the permission of operating the hospital automatic guided vehicle to the second tagged device.

12. The hospital automatic guided vehicle of claim 11, wherein:
the receiver is further configured to receive inventory data of another hospital automatic guided vehicle; and
the hospital automatic guided vehicle further comprises a display device, movably attached to the vehicle housing, coupled to the controller, and configured to display the inventory data of another hospital automatic guided vehicle.

13. A method of operating a hospital automatic guided vehicle comprising a vehicle housing, an object tracking sensor, a plurality of wheels and a controller, the method comprising:
the object tracking sensor tracking a location of a short-range wireless transmitter; and
the controller controlling the plurality of wheels to move the hospital automatic guided vehicle according to the location of the short-range wireless transmitter.

14. The method of claim 11, wherein the hospital automatic guided vehicle further comprises a drawer comprising an electronic lock, and the method further comprises:
the controller setting a permission to access the drawer for a tagged device containing an identifier;
the electronic lock scanning the identifier from the tagged device; and
the electronic lock unlocking the drawer when the identifier identifies a tagged device authorized to access the drawer.

15. The method of claim 13, wherein the hospital automatic guided vehicle further comprises a position sensor, memory, and a receiver, and the method further comprises:
the position sensor determining a location of the hospital automatic guided vehicle; and
the receiver receiving healthcare data according to the location of the hospital automatic guided vehicle and storing the healthcare data in the memory.

16. The method of claim 15, wherein the hospital automatic guided vehicle further comprises a transmitter, and the method further comprises the transmitter transmitting the healthcare data to a remote server.

17. The method of claim 13, wherein the hospital automatic guided vehicle further comprises a proximity sensor, and the method further comprises:
a proximity sensor detecting a space for movement of the hospital automatic guided vehicle; and
when the space for movement of the hospital automatic guided vehicle is less than a predetermined space, the controller controlling at least one of the plurality of wheels to rotate by an angle and proceed to move the hospital automatic guided vehicle.

18. The method of claim 13, further comprising:
the controller setting a permission of operating the hospital automatic guided vehicle for a first tagged device containing a first identifier.

19. The method of claim 18, wherein the hospital automatic guided vehicle further comprises a receiver and an input device, and the method further comprises:
the receiver receiving a permission reset instruction indicating a request for resetting the permission of operating the hospital automatic guided vehicle to a second tagged device containing a second identifier; and
the input device receiving a confirmation instruction indicating a confirmation of resetting the permission of operating the hospital automatic guided vehicle to the second tagged device containing the second identifier.

20. The method of claim 19, wherein the hospital automatic guided vehicle further comprises a display device, and the method further comprises:
the receiver receiving inventory data of another hospital automatic guided vehicle; and
the display device displaying the inventory data of another hospital automatic guided vehicle.
